(19) United States
(12) Patent Application Publication
(10) Pub. No.: US 2025/0135215 A1
(43) Pub. Date: May 1, 2025

(54) NON-TRANSVENOUS ICD SYSTEM WITH AT LEAST THREE SENSING ELECTRODES AND SELECTABLE SENSING VECTORS

(71) Applicant: BIOTRONIK SE & Co. KG, Berlin (DE)

(72) Inventors: Thomas DOERR, Berlin (DE); Ingo WEISS, Berlin (DE)

(73) Assignee: BIOTRONIK SE & Co. KG, Berlin (DE)

(21) Appl. No.: 18/837,793

(22) PCT Filed: Feb. 2, 2023
(86) PCT No.: PCT/EP2023/052574

§ 371 Date: Aug. 12, 2024

(30) Foreign Application Priority Data
Feb. 24, 2022 (EP) 22158525.0

(51) Int. Cl. A61N 1/39; A61B 5/341; A61N 1/05

(52) U.S. Cl. CPC A61N 1/3956; A61B 5/341; A61N 1/0504

(57) ABSTRACT

A non-transvenous ICD system comprises a defibrillation generator, a controller, an electrode line, a shock electrode arranged at the electrode line and at least three sensing electrodes arranged at various positions within the ICD system. The defibrillation generator is configured for generating electric shocks and applying the electric shocks to cardiac tissue of a patient via the shock electrode. The controller is configured for
(i) sensing plural signals indicating a cardiac activity by detecting an electric voltage along each of a plurality sensing vectors, wherein each sensing vector extends between two of the sensing electrodes,
(ii) automatically selecting at least one of the sensed signals based on predetermined criteria being fulfilled, and
(iii) controlling the defibrillation generator for generating the electric shocks based on the at least one selected signal.

### CROSS-REFERENCE TO RELATED APPLICATIONS

This application is the United States National Phase under 35 U.S.C. § 371 of PCT International Patent Application No. PCT/EP2023/052574, filed on Feb. 2, 2023, which claims the benefit of European Patent Application No. 22158525.0, filed on Feb. 24, 2022, the disclosures of which are hereby incorporated by reference herein in their entireties.

### TECHNICAL FIELD

The present invention relates to a non-transvenous ICD (implantable cardioverter defibrillator) system and to a method for controlling a generation of electric shocks in a non-transvenous ICD system.

### BACKGROUND

An ICD is a miniaturized device implantable inside a body of a patient suffering from cardiac insufficiencies. The ICD is able to perform cardioversion, defibrillation, and, optionally, pacing of the patient's heart. The device is therefore capable of correcting most life-threatening cardiac arrhythmias. Accordingly, the ICD may provide treatment and prophylactic therapy for patients at risk for sudden cardiac death due to ventricular fibrillation and ventricular tachycardia.

Generally, a conventional ICD comprises two components. On the one hand, an ICD main device, which comprises of a control unit, a battery and an electrode, and a venous electrode, which is anchored in a right ventricle of the patient's heart. The ICD is typically implanted under the skin in front of or in the left chest muscle. A diagnostic part detects disorders requiring treatment by permanent ECG lead. The diagnostic part uses sensing electrodes for sensing a cardiac activity of the patient's heart. A stimulation part then triggers a generation of an electric shock. The electric shock is generated by a defibrillation generator and is applied to tissue of the patient at or close to the heart using a shock electrode. During the shock, an electric field is established. In modern devices, this can be established either between a venous shock electrode and the housing of the ICD device or, if so-called dual-coil electrodes are used, between two separate shock electrodes, i.e., coils, of the venous electrode. Thus, depending on the individual anatomy of the patient, the electric field may be optimized with the goal of capturing as much myocardial tissue as possible in the electric field.

A housing of an ICD is typically made of tissue-compatible material such as titanium. Encapsulated inside are a microcomputer with electronic circuitry and a long-life battery. On top of the housing, there is generally a header comprising connectors for the probes (electrodes) that are inserted into the heart. During operation, cardiac signals are constantly transmitted to the ICD's microcomputer from sensing probes located at the ends of the probes. If the incoming signals are identified by the computer program, for example, as ventricular flutter or fibrillation, an integrated defibrillation electrode delivers shock-like pulses to the ventricle until the heart rhythm stabilizes to the programmed normal values.

Whereas with the conventional ICD the tip of the intravenous electrode must generally be advanced directly into the ventricle, which requires fluoroscopy, this is not necessary with the insertion of a non-transvenous ICD. In contrast to the conventional ICD, such non-transvenous ICD is typically implanted subcutaneously or submuscularly, i.e., its electrodes are placed under the skin typically in a region next to the sternum. This makes implantation easier and without radiation exposure and may reduce a risk of infection or complications associated with transvenous electrodes. However, stronger and more frequent electrical shocks are generally required to terminate an arrhythmia of the heart and prevent impending cardiac arrest compared to the conventional ICD.

A disadvantage of the non-transvenous ICD system may be a comparatively poor perception performance, so that inadequate shock therapies and delayed therapies may occur. Furthermore, about 10% of patients cannot be treated with this system because they do not meet criteria for ECG screening before implantation, i.e., available ECG vectors do not fulfil the signal conditions for a correct perception function. This disadvantage may be exacerbated by the fact that locations of the sensing electrodes forming perception poles can generally not be varied, since both the implantation location of an electrode line including a shock electrode along with perception poles and the implantation location of the generator are typically fixed by the requirements of defibrillation field propagation.

Another disadvantage may be that the housing of the ICD is generally unsuitable for perception for several tens of seconds after a defibrillation shock has been delivered due to afterpotentials, so that only the typically two perception poles on the electrode line may be used after a shock has been delivered and thus only a single perception vector extending between the sensing electrodes forming these perception poles is available for this time.

International Publication No. WO 2018/005373 A1, International Publication No. WO 2018/093605 A1, U.S. Publication No. 2018/0185660 A1 and U.S. Publication No. 2015/0216433 A1 relate to cardiac therapy systems.

There may be a need for a non-transvenous ICD system and a method for generating electric shocks in a non-transvenous ICD system enabling an improved perception performance. Such improved perception performance may increase a number of patients for which a non-transvenous ICD therapy is applicable due to a positive ECG screening.

Such needs may be met with the subject-matter of the independent claims. Advantageous embodiments are defined in the dependent claims as well as the corresponding specification and figures.

The present disclosure is directed toward overcoming one or more of the above-mentioned problems, though not necessarily limited to embodiments that do.

### SUMMARY

According to a first aspect of the present invention, a non-transvenous ICD system is proposed. The system comprises a defibrillation generator, a controller, an electrode line, a shock electrode arranged at the electrode line and at least three sensing electrodes arranged at various positions within the ICD system. The defibrillation generator is configured for generating electric shocks and applying the electric shocks to cardiac tissue of a patient via the shock electrode. The controller is configured for executing at least the following functionalities, preferably in the indicated order:
(i) sensing plural signals indicating a cardiac activity by detecting an electric voltage along each of a plurality sensing vectors, wherein each sensing vector extends between two of the sensing electrodes,
(ii) automatically selecting at least one of the sensed signals based on predetermined criteria being fulfilled, and
(iii) controlling the defibrillation generator for generating the electric shocks based on the at least one selected signal.

Briefly summarised in a non-limiting manner, embodiments of the present invention relate to a non-transvenous ICD system in which, additionally to a shock electrode being arranged at an electrode line and being connected to a defibrillation generator, at least three sensing electrodes are provided at positions spaced to each other. Therein, each pair of sensing electrodes forms a sensing vector. Accordingly, electric voltages may be sensed along each of a plurality of different sensing vectors and signals indicating a cardiac activity may be determined based on the detected electric voltages for each of the sensing vectors. As there is a multiplicity of resulting signals available from each of the multiple sensing vectors, the controller of the ICD is configured for automatically selecting at least one of the sensed signals which appears to be suitable as a basis for controlling the generation of the electric shocks by the defibrillation generator. Therein, such automatic selection is realised by determining whether or not the signal fulfils predetermined criteria, examples of which being discussed in more detail further below. The selection of the signals may be adapted such as to enable diagnostic statements and/or therapy decisions. Due to such specific automatic selection of signals sensed by one or more of a plurality of sensing vectors, a most suitable, i.e., for example, a most reliable or most characteristic, signal may be selected amongst the signals provided by the various sensing vectors and as a result, a perception quality of the ICD system may be improved. Furthermore, a reliability of the ICD system may be increased and/or a construction of the ICD system may be simplified as a number of components included in the ICD system may be reduced.

In the following, characteristics of embodiments of the present invention will be described in more detail.

As major components, the ICD system may comprise a housing and an electrode line extending from the housing. In the housing, the defibrillation generator and the controller are accommodated together with further components such as a battery, connectors and/or further circuitry. The electrode line, sometimes also referred to as electrode lead, carries one or more shock electrodes, sometimes also referred to as shock coils. Furthermore, one or more sensing electrodes, sometimes also referred to as perception poles, are generally provided at the electrode line. Additionally, one or more sensing electrodes may be provided at or close to the housing of the ICD. The sensing electrodes may be separate components such as ring electrodes, sheet electrodes, coil electrodes, etc. carried by other components of the ICD. Alternatively, the sensing electrodes may be realised as being integrated into other components of the ICD, for example, as an integral part of the shock electrode, of the housing, of a header at the housing or of a connector of the electrode line.

For example, one or more sensing electrodes may be arranged at the electrode line proximal to the shock electrode and/or one or more sensing electrodes may be arranged at the electrode line distal to the shock electrode. Generally, the sensing electrodes may be arranged at any position along the electrode line and at any distance with regards to the shock electrode. For example, the sensing electrodes may be arranged at a distance of up to 20 mm or at a distance of more than 30 mm from the nearest shock electrode.

The electrode line may be configured such as to be implanted into the thorax of the patient in a configuration in which a proximal portion of the electrode line extends approximately parallel to the ribs whereas a distal portion of the electrode line extends approximately parallel to the sternum. The shock electrode is typically arranged at the distal portion. Furthermore, at least one of the sensing electrodes may be arranged at the distal portion. For example, one or more sensing electrodes may be provided distally to the shock electrode and/or one or more sensing electrodes may be provided proximally to the shock electrode but still within the distal portion of the electrode line. Furthermore, one or more sensing electrodes may be provided at the proximal portion of the electrode line. For example, one of the sensing electrodes may be provided at a position at the electrode line being closer than 50 mm from an electrode plug at which the electrode line is connected to a socket provided at the header of the housing of the ICD device.

The sensing electrode may be implemented as a ring electrode and may have a length in a range of, e.g., between 0.5 to 5 times its diameter. Alternatively, the sensing electrode may be implemented as a coil having a length of more than 3 times its diameter.

The sensing electrode may be isodiametric with regards to the electrode line. In other words, an outermost surface of the sensing electrode may be flush with an outermost surface of the electrode line in an area adjacent to the sensing electrode. Accordingly, preferably no steps or other transitions in diameters are present at the outer surface of the electrode line including the sensing electrodes. Such smooth and homogeneous circumferential surface of the electrode line may simplify explanting the electrode line, for example, in cases where the ICD system has to be removed from the patient's body.

The electrode line or optionally the entire ICD system may be configured for subcutaneous or submuscular implantation. I.e., materials, geometries and/or functionalities of the electrode line or the ICD system may be adapted such that the respective component may be easily and reliably implanted into the patient's body. Particularly, the electrode line may be configured for substernal implantation.

Each two, i.e., each pair, of such sensing electrodes forms a sensing vector. A distance between the two sensing electrodes defines a length of the sensing vector. A virtual line between the two sensing electrodes defines an orientation or a direction of the sensing vector. The ICD system is configured for detecting or measuring an electric voltage along each of such sensing vectors. Generally, such electric voltage correlates with a cardiac activity, i.e., a current motion status of the patient's heart. Due to the fact that the various sensing vectors have different lengths and/or orientations, the correlation between the cardiac activity and the electric voltage measured at one of the sensing vectors typically varies. It has been found that, in certain conditions, an electric voltage measured at a first sensing vector may be most suitable for evaluating a cardiac activity whereas in other conditions, an electric voltage measured at another second sensing vector may be more suitable for such purpose. Accordingly, by automatically suitably selecting one of the signals sensed at the plural sensing vectors, an overall perception performance of the ICD system may be increased by controlling the defibrillation generator based on the most suitable selected signal(s) for generating the electric shocks.

According to an embodiment, the controller is configured for automatically selecting the at least one of the sensed signals based on an evaluation of characteristics indicated by the signal.

The characteristics may include one or more of the following options:
a ratio of an R-wave amplitude and a T-wave amplitude,
a signal-to-noise ratio,
time-dependent variations of the signal-to-noise ratio,
time-dependent variations of amplitudes,
deviations of amplitudes from reference values,
time-dependent variations of morphologic variations, and
deviations of morphologic variations from reference values.

In other words, the controller may sense the signals by detecting the electric voltage occurring at each of the various sensing vectors and may then evaluate specific characteristics in such signals.

For example, as the detected electric voltage correlates with the cardiac activity of the heart, specific waveforms typically occur in a plot of the voltage amplitude, such waveforms including generally an R-wave and a T-wave. A ratio of the amplitudes of such R-wave and T-wave generally correlates with a quality of the signal detected at the sensing vector and may therefore be used for discriminating between more suitable and less suitable sensing vectors.

Similarly, a signal-to-noise ratio (SNR) as well as time-dependent variations of such SNR in the detected electric voltage signal generally correlates with a signal quality.

As a further alternative, time-dependent variations of amplitudes occurring during detecting the electric voltage at the sensing vector or deviations of such amplitudes from reference values may also correlate with a signal quality.

As even a further alternative, time-dependent variations of morphologic variations such as a width of a peak, a surface area along a typical waveform, etc. as well as deviations of such morphologic variations from reference values typically correlate with a signal quality.

By automatically suitably selecting one of the signals sensed at the plural sensing vectors by suitably evaluating its characteristics, the overall perception performance of the ICD system may be improved.

According to an embodiment, the controller is configured for automatically evaluating the sensed signals based on at least one condition including a body position of the patient, an activity status of the patient, a time of day and a therapy condition.

In other words, upon determining which of the sensed signals is to be selected for controlling the generation of the electric shocks by the defibrillation generator, various external conditions may be taken into account.

For example, a current position of the patient's body may be monitored. Such body position may correlate with the cardiac activity of the patient. For example, it may be monitored whether the patient is staying, sitting or lying. The body position may correlate with an orthostatism. The body position may be detected with one or more position sensors arranged at the patient's body, with a camera observing the patient's body or with any other technical means.

Alternatively or additionally, the activity status of the patient may be monitored. Such activity status typically correlates with the cardiac activity of the patient. For example, it may be monitored whether the patient is currently active or passive and, optionally, a degree of activity may be determined. The activity status may be detected, for example, with one or more motion sensors arranged at the body of the patient, with a camera observing the patient or with any other technical means.

As a further alternative or addition, the time of the day may be taken into account. Generally, the patient's cardiac activity varies during the day and depends on the time of the day, for example, in accordance with a circadian rhythm. The time of the day may be detected, for example, with a clock or a chronometer.

As even a further alternative or addition, a therapy condition may be monitored and may be taking into account upon selecting the sensed signals. Typically, a cardiac activity correlates with such therapy condition. For example, the cardiac activity during pacing the heart differs from the cardiac activity in a non-paced condition. Furthermore, the cardiac activity in a state immediately after a shock substantially differs from the normal cardiac activity. As another example, the cardiac activity typically correlates with a body temperature.

According to an embodiment, the controller is configured for automatically selecting the at least one of the sensed signals based on a comparison of a characteristics indicated by the signal with a predetermined limit value.

Expressed differently, for selecting a signal, the controller may determine value indicating characteristics of each signal sensed at the various sensing electrodes and may compare this characteristics value with an associated predetermined limit value. Based on such comparison, a most suitable signal or a few most suitable signals may be easily determined for subsequently controlling the defibrillation generator.

According to a further specified embodiment, the signal is selected based on comparison results indicating at least one of the following characteristics:
a ratio of an R-wave amplitude and a T-wave amplitude being higher than a predetermined limit value,
a ratio of an R-wave amplitude and a T-wave amplitude varying below a predetermined limit value despite a change of a body position of the patient having occurred,
a signal-to-noise ratio being higher than a predetermined limit value,
time-dependent variations of the signal-to-noise ratio being below a predetermined limit value despite at least one of a change of a body position and an activity of the patient having occurred, or
at least one of signal amplitudes and morphologic metrics varying below a predetermined limit value despite at least one of a change of a body position and an activity of the patient having occurred.

In other words, a sensed signal may be selected as being suitable in case the ratio (i.e., the quotient) of the R-wave amplitude to the T-wave amplitude is larger than a limit value. Such limit value may have been predetermined based, for example, on previous measurements, experiments, simulations, etc. Having such high ratio of amplitude values may indicate a high signal quality. Similarly, a high signal quality may be assumed in case such ratio remains below a predetermined limit value although a position or orientation of the patient's body has just been changed.

Alternatively or additionally, a good signal quality may be assumed in cases where the SNR is larger than a predetermined limit value and/or variations of the SNR over time remain below a predetermined limit value despite the position or orientation of the patient's body has been changed and/or an activity of the patient has been observed.

According to an embodiment, the controller is configured for automatically selecting a different one of the sensed signals upon a shock having been generated by the defibrillation generator.

Expressed differently, the selected signal may be automatically changed as soon as a shock has been applied by the ICD device. Typically, a signal morphology substantially changes upon a shock being applied by the ICT device. Such change may result in a signal provided by another sensing vector being more suitable than the signal provided by a sensing vector used before application of the shock. For example, before application of a shock, a sensing vector may be preferable in which one of the sensing electrodes is provided directly at or adjacent to the housing of the ICD system. However, upon generating and applying a shock, such housing and/or the sensing electrode at the housing may be disturbed due to electric afterpotentials remaining after the shock for several seconds or even tens of seconds. Accordingly, during such period after the shock application, selecting another sensing vector having its sensing electrodes distant to the ICD housing may be preferable.

According to an embodiment, the controller device comprises a switching device, an amplifier device and an evaluation device. The switching device is configured for selectively connecting electrodes associated to each one of the sensing vectors to the amplifier device. The amplifier device is configured for amplifying the signals. The evaluation device is configured for evaluating the amplified signals for controlling the generation of the electric shocks based on the selected signals.

Thus, the control device may comprise several components interacting with each other, each component having its specific function. Therein, the switching device may selectively connect a pair of electrodes of one of the sensing vectors to the amplifier device. The amplifier device may then amplify the signal provided by the sensing vector currently connected thereto. Finally, the amplified signal may be evaluated by the evaluation device and the result of such evaluation may be used for controlling the defibrillation generator to suitably generate the electric shocks. Such separate components and their functionalities may be easily implemented and may provide a reliable operation of the entire controller device.

According to a further specified embodiment, the switching device is configured for selectively connecting to the amplifier device exclusively the electrodes associated to the one of the sensing vectors for which the predetermined criteria are fulfilled.

In other words, only those sensing vectors and their electrodes are selectively connected to the amplifier device for which the signals provided by the sensing vector fulfil the predetermined criteria, whereas other sensing vectors are not connected by the switching device to the amplifier device. Accordingly, a complexity of a switching operation and/or of the switching device may be reduced. Furthermore or alternatively, a complexity of the amplifier device and, particularly, a number of amplification channels may be limited.

According to a further embodiment, the switching device is configured for connecting the electrodes associated to one of the sensing vectors to the amplifier device sequentially for all of the sensing vectors.

Expressed differently, the switching device may connect only the electrodes of a single sensing vector at a given time to the amplifier device. The plural sensing vectors may then be sequentially, i.e., one after the other, connected to the amplifier device. Thereby, a complexity of the amplifier device and, particularly, a number of amplification channels included in the amplifier device may be limited.

According to an embodiment, in a case in which plural of the sensed signals fulfil the predetermined criteria, the controller is configured for automatically selecting the plural sensed signals and controlling the defibrillation generator for generating the electric shocks based on an evaluation of a combined signal in which the plural signals are combined in a weighted manner.

In other words, when plural sensing vectors fulfil the criteria of being suitable for controlling the shock generation, their information content may be combined and may be weighted in such combination before being used for controlling the defibrillation generator. Therein, the weighting of the various signals may correlate with a degree to which the predetermined criteria are fulfilled. Such combining and weighting plural suitable signals may further increase a perception performance of the ICD system.

According to an embodiment, the ICD system comprises at least three sensing electrodes arranged at the electrode line.

Generally, the at least three sensing electrodes of the ICD system may be arranged anywhere within the ICD system, i.e., for example, at the housing, the header or the electrode line. However, it may be preferred to provide the ICD system with at least three sensing electrodes at the electrode line, not excluding that one or more further sensing electrodes are provided at other components such as the housing of the ICD system. Having three sensing electrodes at the electrode line may improve a perception performance of the ICD system. Particularly, it may be preferable to arrange at least one sensing electrode distal to the shock electrode and arrange at least two sensing electrodes proximal to the shock electrode, i.e., between the shock electrode and the housing of the ICD system. Specifically, it may be preferable to provide at least one of these two sensing electrodes closer to the shock electrode than to the ICD housing and to provide the other of the two sensing electrodes closer to the ICD housing than to the shock electrode. Thereby, positions and/or orientations of sensing vectors may be varied in a large range. This may improve the overall perception performance as for different cardiac conditions a suitable sensing vector may be selected by the ICD system automatically.

According to an embodiment, at least one sensing electrode is arranged at a header provided at a housing accommodating the defibrillation generator and the controller.

In other words, the ICD housing typically comprises a header at which other components as specifically the electrode line may be connected electrically and mechanically to the ICD housing. While the ICD housing is typically made from an electrically conductive material such as titanium, the header or at least parts of the header may be electrically isolated from a remainder of the ICD housing. It may be preferable to provide at least one of the sensing electrodes at the header, thereby enabling that this sensing electrode may be electrically isolated or electrically decoupled from the ICD housing. Accordingly, when an electric shock is generated by the defibrillation generator in the ICD housing, such electric shock does not significantly disturb the signals sensed at the sensing vector comprising the sensing electrode at the isolated header.

According to an embodiment, the ICD system comprises at least four sensing electrodes being arranged such that pairs of electrodes provide at least four sensing vectors being non-parallel to each other.

Expressed differently, the ICD system may comprise four or more sensing electrodes. Therein, when the ICD system is implanted in the patient's thorax, these sensing electrodes are not arranged along a line but are distributed to various positions within the thorax. Particularly, no three out of these at least four sensing electrodes are arranged along a line. For example, at least one sensing electrode may be provided distally with regards to the shock electrode and therefore close to a distal end of the electrode line. Furthermore, at least one sensing electrode may be provided proximally with regards to the shock electrode but relatively close to the shock electrode. Additionally, at least one sensing electrode may be provided proximally with regards to the shock electrode but relatively distant from the shock electrode and closer to the housing of the ICD system than to the shock electrode. Finally, at least one sensing electrode may be provided at or close to the housing of the ICD system.

Accordingly, each sensing vector composed of two of the sensing electrodes is not only arranged at a different position but also at a different orientation with respect to other sensing vectors composed of other pairs of sensing electrodes. Thus, none of the sensing vectors extends in parallel to another one of the sensing vectors. Instead, all sensing vectors are nonparallel to each other. Therein, the term “nonparallel” may be interpreted as indicating that two sensing vectors are not substantially parallel to each other, wherein “substantially parallel” may include all orientations in which the sensing vectors include an angle of between −5° to +5° or an angle of between −10° to +10° or even an angle of between −20° to +20°. Due to such nonparallel orientations of the sensing vectors, their signals may correlate with the cardiac activity in substantially different manners. Accordingly, by selecting the most suitable one of the sensing vectors out of the plurality of substantially differently oriented sensing vectors and using the signal provided by such preferred sensing vector, the generation of the electric shocks may be controlled in a beneficial way.

According to a second aspect of the present invention, a method for controlling generating of electric shocks in a non-transvenous ICD system is suggested. Therein, the non-transvenous ICD system comprises a defibrillation generator, a controller, an electrode line, a shock electrode arranged at the electrode line and at least three sensing electrodes arranged at various positions within the ICD system. The defibrillation generator is configured for generating electric shocks and applying the electric shocks to cardiac tissue of a patient via the shock electrode. The method comprises at least the following steps, preferably in the indicated order:
(i) sensing plural signals indicating a cardiac activity by detecting an electric voltage along each of a plurality sensing vectors, wherein each sensing vector extends between two of the sensing electrodes,
(ii) automatically selecting at least one of the sensed signals based on predetermined criteria being fulfilled, and
(iii) controlling the defibrillation generator for generating the electric shocks based on the at least one selected signal.

Embodiments of the proposed method may be implemented upon operating the ICD system when installed in a patient's body. The ICD system may be an embodiment of the above described first aspect of the present invention.

It shall be noted that possible features and advantages of embodiments of the present invention are described herein with respect to various embodiments of a non-transvenous ICD system, on the one hand, and with respect to various embodiments of a method for controlling a generation of electric shocks with a non-transvenous ICD system, on the other hand. One skilled in the art will recognize that the features may be suitably transferred from one embodiment to another and features may be modified, adapted, combined and/or replaced, etc. in order to come to further embodiments of the present invention.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In the following, advantageous embodiments of the present invention will be described with reference to the enclosed drawings. However, neither the drawings nor the description shall be interpreted as limiting the present invention.

FIG. 1 shows an ICD system according to an embodiment of the present invention.

FIGS. 2A-C show cross sectional views of different implementations of headers of ICD systems according to embodiments of the present invention.

FIG. 3 shows a longitudinal sectional view of a header of an ICD system according to an embodiment of the present invention.

FIG. 4 shows a cross sectional view of a header of an ICD system according to an embodiment of the present invention.

FIG. 5 shows a cross sectional view of a header of an ICD system according to an embodiment of the present invention.

The figures are only schematic and not to scale. Same reference signs refer to same or similar features.

### DETAILED DESCRIPTION

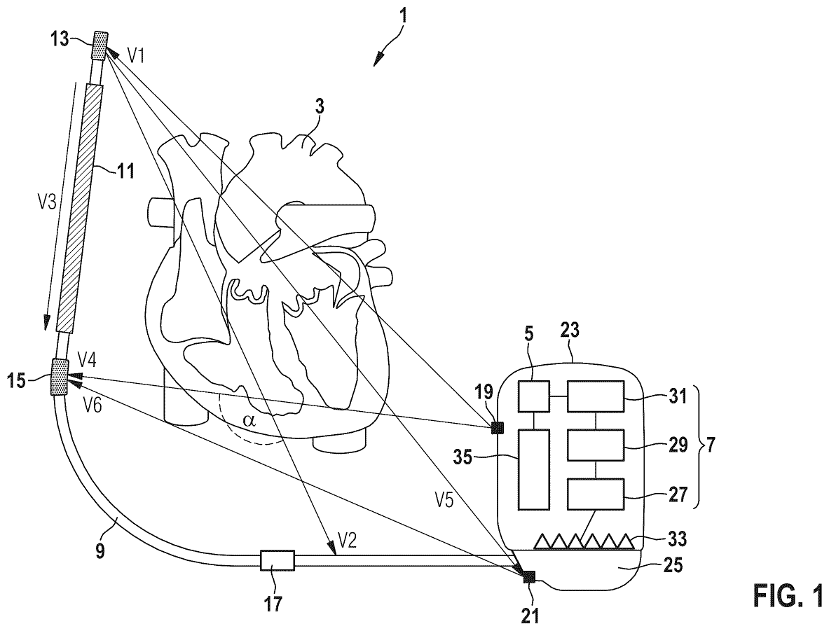

FIG. 1 shows a non-transvenous ICD system 1. The ICD system 1 is configured for being implanted into a thorax of a patient at a location adjacent to the patient's heart 3. The ICD system 1 comprises a defibrillation generator 5, a controller 7, an electrode line 9, a shock electrode 11 arranged at the electrode line 9 and several sensing electrodes 13, 15, 17, 19, 21 arranged at various positions within the ICD system 1.

Several components of the ICD system I are comprised in a housing 23. The housing 23 is generally made from a biocompatible material such as titanium and encloses the components of the ICD system 1 for protecting them against physical and chemical influences. Particularly, the housing 23 accommodates the defibrillation generator 5, the controller 7 and a battery 35. The controller 7 comprises a switching device 27, an amplifier device 29 and an evaluation device 31.

A header 25 is provided at one side of the housing 23. The header 25 serves inter-alia for connecting the electrode line 9 and the shock electrode 11 and the sensing electrodes 13, 15, 17 arranged at this electrode line 9 to the components comprised in the housing 23.

When implanted into the patient's thorax, the ICD system 1 is configured such that the housing 23 is arranged subcutaneously, i.e., directly under the skin of the patient or at a muscle of the patient. The electrode line 9 is then implanted in a curved configuration such as to extend with its proximal portion substantially parallel to the patient's ribs, i.e., in a substantially horizontal direction, and with its distal portion substantially parallel to the patient's sternum, i.e., in a substantially vertical direction.

In the example shown in the figures, three sensing electrodes 13, 15, 17 are arranged at the electrode line 9. Therein, a first sensing electrode 13 is arranged distal to the shock electrode 11. A second sensing electrode 15 is arranged proximal to and close to the shock electrode 11, i.e., at the distal portion of the electrode line 9. A third sensing electrode 17 is arranged proximal to but distant to the shock electrode 11, i.e., at the proximal portion of the electrode line 9 and at a position closer to a proximal end of the electrode line 9 than to the shock electrode 11. A fourth sensing electrode 19 is arranged at the housing 23 of the ICD system 1. A fifth sensing electrodes 21 is arranged at a header 25 at the housing 23.

Each pair of sensing electrodes 13, 15, 17, 19, 21 forms a sensing vector V1, V2, . . . , V6 extending along a linear line between the sensing electrodes 13, 15, 17, 19, 21 of this pair. Preferably, at least four of the sensing electrodes 13, 15, 17, 19, 21 are arranged such that pairs of these sensing electrodes provide at least four sensing vectors V1, V2, . . . . V6 which extend nonparallel to each other, i.e., with an angle a with respect to each other, this angle being, for example, larger than +10°. Each of the sensing vectors V1, V2, . . . , V6 is connected to one of a plurality of sensing vector channels 33. The switching device 27 is configured for selectively connecting the sensing electrodes 13, 15, 17, 19, 21 associated to each of the sensing vectors V1, V2, . . . , V6 at each of the sensing vector channels 33 to the amplifier device 29. The amplifier device 29 may then amplify signals corresponding to electric voltages sensed at these sensing electrodes 13, 15, 17, 19, 21. Finally, the evaluation device 31 may evaluate the amplified signals for controlling the defibrillation generator 5 to generate electric shocks based on the amplified signals. These electric shocks are then supplied to the shock electrode 11 in order to generate a strong electric field in a neighbourhood of the heart 3 and to thereby stimulate muscular tissue of the heart 3 in a defibrillation action.

In order to enable a highly reliable detection of a cardiac activity of the heart 3, the controller 7 of the ICD system 1 is specifically configured for automatically selecting one or more best suitable ones of the sensing vectors V1, V2, . . . , V6 in order to then control the defibrillation generator 5 based on the signals provided by these selected sensing vectors. Particularly, the controller 7 is configured for sensing plural signals indicating the cardiac activity by detecting electric voltages along each of the plurality of sensing vectors V1, V2, . . . , V6. For such purpose, the switching device 27 may, for example, connect the sensing electrodes 13, 15, 17, 19, 21 associated to one of the sensing vectors V1, V2, . . . , V6 to the amplifier device 29 in a sequential manner. It may then be automatically checked whether the signals provided by each of the sensing vectors V1, V2, . . . , V6 fulfil predetermined criteria. Based on such checking action, one or more most suitable ones of the sensing vectors V1, V2, . . . , V6 may be automatically selected. The sensing electrodes 13, 15, 17, 19, 21 of the selected one or more sensing vectors V1, V2, . . . . V6 may then be selectively connected by the switching device 27 to the amplifier device 29. After amplification of the signals sensed at these sensing vectors V1, V2, . . . , V6, the defibrillation generator 5 may be controlled based on the evaluation of the amplified signals by the evaluation device 31.

In cases in which more than one sensing vector V1, V2, . . . , V6 fulfils the predetermined criteria, the controller 7 automatically selects the plural signals sensed at these sensing vectors V1, V2, . . . , V6 and combines these plural signals in a weighted manner. The defibrillation generator may then be controlled based on such combined signals. The weighting of the individual signals may be based on a degree to which the predetermined criteria are fulfilled.

In order to check whether the signals sensed at the sensing vectors V1, V2, . . . , V6 fulfil the predetermined criteria, the controller 7 is configured for evaluating characteristics indicated by such signals. Optionally, in such evaluation process, further conditions may be taken into account, such further conditions including, for example, a current position of a body of the patient, an activity status of the patient, a time of day and/or a therapy condition.

For example, it may be evaluated whether a ratio of an R-wave amplitude and a T-wave amplitude represented in such sensor signal is higher than a predetermined limit value and/or whether such ratio varies below a predetermined limit value despite a change of a body position of the patient having been observed. Alternatively or additionally, it may be evaluated whether a signal-to-noise ratio is higher than a limit value and/or whether time-dependent variations of such SNR remain below a predetermined limit value despite a change of a body position and/or an activity of the patient having been observed. As a further additional or alternative option, it may be evaluated whether at least one of signal amplitudes or morphologic metrics vary below a predetermined limit value despite a change of the body position and/or an activity of the patient having been observed.

As another option, the controller 7 may be configured for automatically selecting the sensed signal provided by another one of the sensing vectors V1, V2, . . . , V6 as soon as an electric shock has been/is applied by the defibrillation generator 5. In other words, when it is determined that a defibrillation shock has been generated, the controller 7 may automatically switch from a former suitable one of the sensing vectors V1, V2, . . . , V6 to another one of the sensing vectors V1, V2, . . . , V6. This particularly applies in a case where the former sensing vector V1, V2, . . . , V6 comprised the fourth sensing electrode 19 arranged at the housing 23, as such fourth sensing vector 19 is generally substantially disturbed by the applied electric shock and may therefore not reliably sense any electric voltages for at least some seconds. Due to such automatic switching to another sensing vector V1, V2, . . . , V6, the ICD system 1 may bridge such “blinded” period after the application of a shock by temporarily switching to the other sensing vector.

Hereafter, some possible implementations of features and characteristics of embodiments of the ICD system 1 are described in an exemplary manner.

In one implementation variant, the sensing electrodes 13, 15, 17, 19, 21 forming electrode poles (incl. the one or more shock electrodes 11 forming shock coils) are all routed to a same connector via their supply lines. The connector may be designed in accordance with the DF4 standard.

In a further implementation variant, only the electrode poles of the perception poles are routed to a first connector (based on IS4), the shock coils are also routed separately to a HV-capable connector. Preferably, in the case of only one shock coil, this is contacted by means of a DF1 connector. The separation in the HV and perception contacts has the advantage of being able to realize HV distances more easily. Furthermore, the separation of the connectors gives more flexibility in the arrangement.

The electrode line 9 forming the electrode lead or the entire ICD system 1 is preferably MRI compatible.

Furthermore, the system may have a home monitoring connection to remotely transmit signals recorded with the participation of the perception poles.

As visualised in FIG. 1, the ICD system 1 comprises a plurality of sensing vectors forming perception vectors, wherein six exemplary sensing vectors V1-V6 are visualised:
V1: between the fourth sensing electrode 19 at the housing 23 and the distal first sensing electrode 13.
V2: between the first sensing electrode 13 and the most proximal third sensing electrodes 17.
V3: between distal first sensing electrode 13 and the less proximal second sensing electrode 15.
V4: between the fourth sensing electrode 19 at the housing 23 and the less proximal second sensing electrode 15 close to the shock electrode 11.
V5: between the distal first sensing electrode 13 and the fifth sensing electrode 21 at the header 25.
V6: between the fifth sensing electrode 21 at the header 25 and the less proximal sensing second electrode 15.

Based on the predetermined criteria, the sensing vectors may be changed by a user (e.g., a physician) or algorithmically by the implant, even during operation. In particular, this also enables an adaptation to anatomical conditions and a specific implantation of the system (even subsequently).

The sensing vectors V2, V5, V6 or other sensing vectors (not shown) enabled by the provision of additional sensing electrodes at the proximal portion of the sensing line 9 or at the header 25 may be used optionally, e.g., for the discrimination of interfering signals etc.

Due to post-potentials or after-potentials after a defibrillation shock delivery, the housing 23 of the generator may not be used as a perception pole for some tens of seconds, so that, temporarily, sensing vectors V1 and V4 may be disturbed and may therefore not be suitable for sensing cardiac activity. With the additional sensing vectors V2, V3, V5, V6 forming alternative perception poles, at least one sensing vector is available for the redetection phase after shock delivery.

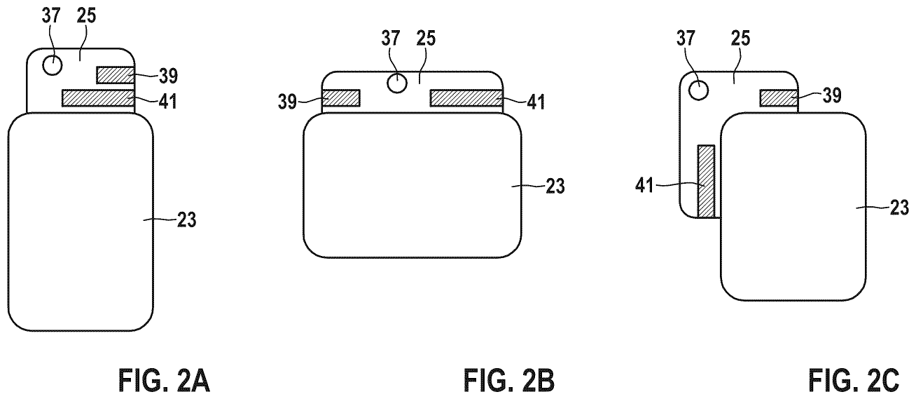

FIG. 2 shows variants of header connector arrangements with separate connectors for HV and perception. The connectors are called first connector 39 and second connector 41 without specifying which is the one for HV or perception. The header 25 further includes at least one mounting hole 37.

As visualised in FIGS. 2A-C, a first connector 39 and a second connector 41 forming sockets may be arranged in the header 25 as follows:
One above the other (access can be from the same or opposite sides).
On (substantially) the same axis. Access then is possible from opposite sides. This is preferable when the header 25 is on the long side of the enclosure.
Around a corner. This means that the tip angle between the axes of the sockets is greater than 5.

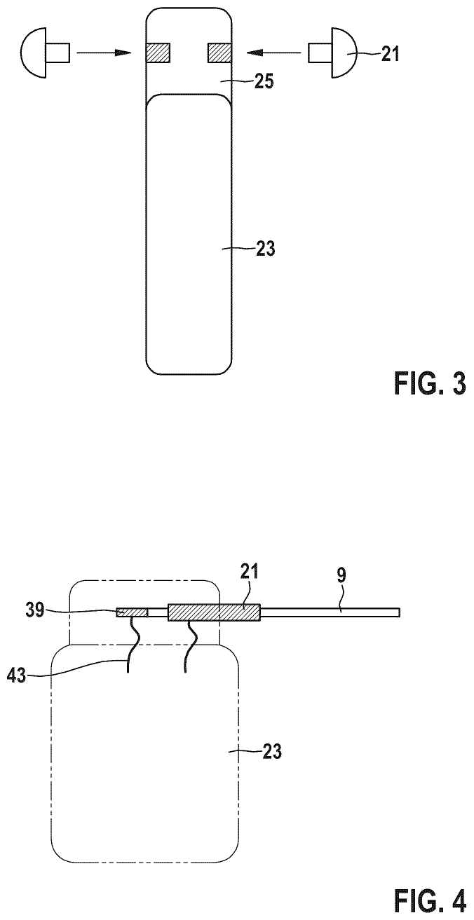

As visualised in parts in FIGS. 3 to 5, an embodiment of the ICD system I may be implemented with
a defibrillation generator 5 with a perception and therapy unit (connected or to be connected),
at least one sensing line 9 forming a combined defibrillation and sensing electrode lead having at least one shock electrode 11 for delivering defibrillation shocks and at least one sensing electrode 13, 15, 17 remote from the housing 23 (additional such sensing electrodes are optional and there is no restriction on location forward or aft of the shock electrode 11)
a header 25 forming a terminal block, the header 25 being electrically insulated from the housing 23 for the at least one electrode lead, and
at least one sensing electrode 21 mounted on the header 25, which is connected to the perception unit and spans at least one sensing vector V5, V6 with at least one of the other sensing electrodes 13, 15, 17 at the electrode line 9.

The sensing electrode 21 mounted on the header 25 is referred to hereinafter as header electrode 21.

The header electrode 21 may be mounted flat on the header 25. Alternatively, the header electrode 21 may be curvedly applied to the header 25. The header electrode 21 may be raised (for better tissue contact) and may be realized, for example, in a mushroom shape. A ground plan may also be any polygonal shape with rounded edges (“mushroom” with angular/oblique ground plan).

The header electrode 21 may be anchored to the header block by casting, for which purpose it optionally has anchor hooks. The header electrode 21 may be connected to the header 25 by screwing, bayonet lock or similar. An electrical connection may be made by welding (laser, resistance), soldering (esp. also hard soldering), screws, rivets, conductive bonding, clamping/spring contact, etc. In a preferred embodiment, it is screwed into a contact block like a grub screw.

Optionally, the header electrode 21 may not be mounted until implantation. Furthermore, optionally, there are several places where it can be mounted to position it on a favorable side depending on the implantation position (as shown in FIG. 3).

The header electrode 21 may be designed as a non-insulated section of the plug of an electrode lead connected to the housing 23 via through contacts 43 (as shown in FIG. 4).

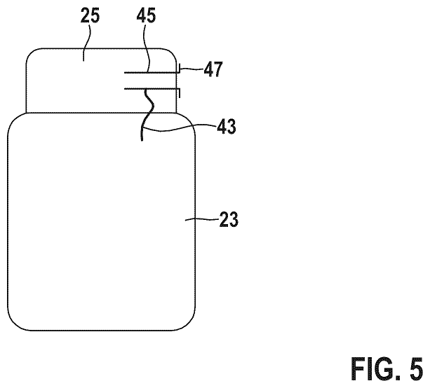

The header electrode 21 may be designed as a conductive guide sleeve 45 for the electrode plug and optionally has a collar or flange 47 around the socket entrance (as shown in FIG. 5). The sleeve 45 may be mounted and arranged such that it comes into contact with body tissue upon the ICD system 1 being implanted. For example, the sleeve 45 order flange 47 may protrude from the header 25.

In case several such sensing electrodes 21 forming several poles are provided, the following alternatives are possible:
The sensing electrodes 21 are separately coupled to one input each of the perception unit.
The sensing electrodes 21 are partially already electrically coupled to each other in the header 25 and are jointly coupled to an input of the perception unit.
The sensing electrodes 21 individually coupled to the perception unit can be interconnected by the perception unit via a switch matrix. This allows the active area and thus the signal characteristics to be adjusted during operation.

Preferred materials for realizing the header electrode 21 are electrically conductive biocompatible materials with a conductivity >1 S/m. Preferred biocompatible metals include PtIr, MP35N, Ti, stainless steel (e.g., 316L).

Optionally, surface enlargement for better lower cut-off frequency for sensing may be achieved, e.g., by
a.) coating with PtIr (e.g., fractal coated (e.g., sputter process)) or Ti coated with TiN;
b.) structuring using, e.g., etching, laser treatment and/or mechanical roughening (e.g., sandblasting).

The first and/or second sensing electrodes 13, 15 located distally or proximally of the shock electrode 11 may be either arranged at a maximum distance of 20 mm from the respective end of the shock electrode 11 or at a minimum distance of 30 mm from the shock electrode 11.

The electrode line 9 is intended for subcutaneous implantation, possibly for substernal implantation. The electrode line 9 or the entire ICD system may be MRI compatible. The ICD system 1 may have a home monitoring connection to remotely transmit such signal recorded with the pole according to the present invention.

Finally, it should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 ICD system
3 heart
5 defibrillation generator
7 controller
9 electrode line
11 shock electrode
13 first sensing electrode
15 second sensing electrode
17 third sensing electrode
19 fourth sensing electrode
21 fifth sensing electrode
23 housing
25 header
27 switching device
29 amplifier device
31 evaluation device
33 sensing vector channel
35 battery
37 fixation hole
39 first connector
41 second connector
43 through contact
45 guiding sleeve
47 flange
V1-V6 sensing vectors

1. A non-transvenous ICD system comprising:
a defibrillation generator,
a controller,
an electrode line,
a shock electrode arranged at the electrode line,
at least three sensing electrodes arranged at various positions within the ICD system,
wherein the defibrillation generator is configured for generating electric shocks and applying the electric shocks to cardiac tissue of a patient via the shock electrode,
wherein the controller is configured for
(i) sensing plural signals indicating a cardiac activity by detecting an electric voltage along each of a plurality sensing vectors, wherein each sensing vector extends between two of the sensing electrodes,
(ii) automatically selecting at least one of the sensed signals based on predetermined criteria being fulfilled, and
(iii) controlling the defibrillation generator for generating the electric shocks based on the at least one selected signal.

2. The system of claim 1, wherein the controller is configured for automatically selecting the at least one of the sensed signals based on an evaluation of characteristics indicated by the signal, the characteristics including at least one of
a ratio of an R-wave amplitude and a T-wave amplitude,
a signal-to-noise ratio,
time-dependent variations of the signal-to-noise ratio,
time-dependent variations of amplitudes,
deviations of amplitudes from reference values,
time-dependent variations of morphologic variations, and
deviations of morphologic variations from reference values.

3. The system of claim 1, wherein the controller is configured for automatically evaluating the sensed signals based on at least one condition including
a body position of the patient,
an activity status of the patient,
a time of day, and
a therapy condition.

4. The system of claim 1, wherein the controller is configured for automatically selecting the at least one of the sensed signals based on a comparison of a characteristics indicated by the signal with a predetermined limit value.

5. The system of claim 4, wherein the signal is selected based on comparison results indicating at least one of
a ratio of an R-wave amplitude and a T-wave amplitude being higher than a predetermined limit value,
a ratio of an R-wave amplitude and a T-wave amplitude varying below a predetermined limit value despite a change of a body position of the patient having occurred,
a signal-to-noise ratio being higher than a predetermined limit value,
time-dependent variations of the signal-to-noise ratio being below a predetermined limit value despite at least one of a change of a body position and an activity of the patient having occurred,
at least one of signal amplitudes and morphologic metrics varying below a predetermined limit value despite at least one of a change of a body position and an activity of the patient having occurred.

6. The system of claim 1, wherein the controller is configured for automatically selecting a different one of the sensed signals upon a shock having been generated by the defibrillation generator.

7. The system of claim 1,
wherein the controller comprises a switching device, an amplifier device and an evaluation device,
wherein the switching device is configured for selectively connecting sensing electrodes associated to each one of the sensing vectors to the amplifier device,
wherein the amplifier device is configured for amplifying the signals,
wherein the evaluation device is configured for evaluating the amplified signals for controlling the generating of the electric shocks based on the selected signals.

8. The system of claim 7, wherein the switching device is configured for selectively connecting to the amplifier device exclusively the sensing electrodes associated to the one of the sensing vectors for which the predetermined criteria are fulfilled.

9. The system of claim 1, wherein the switching device is configured for connecting the sensing electrodes associated to one of the sensing vectors to the amplifier device sequentially for all of the sensing vectors.

10. The system of claim 1,
wherein a plural of the sensed signals fulfil the predetermined criteria,
wherein the controller is configured for automatically selecting the plural sensed signals and controlling the defibrillation generator for generating the electric shocks based on an evaluation of a combined signal in which the plural signals are combined in a weighted manner.

11. The system of claim 1, wherein at least three sensing electrodes are arranged at the electrode line.

12. The system of claim 1, wherein at least one sensing electrode is arranged at a header provided at a housing accommodating the defibrillation generator and the controller.

13. The system of claim 1, wherein the system comprises at least four sensing electrodes being arranged such that pairs of sensing electrodes provide at least four sensing vectors being non-parallel to each other.

14. A method for controlling generating of electric shocks in a non-transvenous ICD system, the non-transvenous ICD system comprising:
a defibrillation generator,
a controller,
an electrode line,
a shock electrode arranged at the electrode line,
at least three sensing electrodes arranged at various positions within the ICD system,
wherein the defibrillation generator is configured for generating electric shocks and applying the electric shocks to cardiac tissue of a patient via the shock electrode
wherein the method comprises:
(i) sensing plural signals indicating a cardiac activity by detecting an electric voltage along each of a plurality sensing vectors, wherein each sensing vector extends between two of the sensing electrodes,
(ii) automatically selecting at least one of the sensed signals based on predetermined criteria being fulfilled, and
(iii) controlling the defibrillation generator for generating the electric shocks based on the at least one selected signal.
